(19) United States
(12) Patent Application Publication
(10) Pub. No.: US 2019/0177588 A1
(43) Pub. Date: Jun. 13, 2019

(54) Compositions, Methods, and Systems For Producing Flocculent Materials for Special Effects

(71) Applicant: XOTRAMORPHIC, LLC, Los Angeles, CA (US)

(72) Inventor: David Hugh Webber, Los Angeles, CA (US)

(73) Assignee: Xotramorphic LLC, Los Angeles, CA (US)

(21) Appl. No.: 16/215,435

(22) Filed: Dec. 10, 2018

Related U.S. Application Data
(60) Provisional application No. 62/597,810, filed on Dec. 12, 2017.

(51) Int. Cl. C09K 3/24; C07C 229/08; B05B 1/24; A63J 5/02

(52) U.S. Cl. CPC C09K 3/24; A63J 5/02; B05B 1/24; C07C 229/08

(57) ABSTRACT

The present invention relates to compositions, methods, and systems for the production of flocculent material. The composition of the invention contains at least one standard or non-standard amino acid, and optionally contains one or more sublimable or evaporable dyes. Flocculent material is made by heating the composition until a vapor is produced, and cooling the heated composition to produce a flocculent material. The system for producing flocculent material includes the composition, a heat source; and one or more walls surrounding the heat source.

### CROSS REFERENCE TO RELATED APPLICATIONS

This application claims the benefit of U.S. Provisional Patent Application No. 62/597,810, titled “Method for Producing Flocculent Materials for Special Effects” filed on Dec. 12, 2017, the contents of which are incorporated herein by reference in its entirety.

### BACKGROUND

Special effects have been produced for years in the movie, theater, and related industries, including special effects which simulate falling, drifting, or lying snow. There are many commercially available machines for producing special effects. For example, one type of available snow machine artificially simulates the appearance of snowflakes by discharging small pieces of aqueous liquid-based foam. However, the foam particles produced by these machines are relatively dense which limits their application for blizzard-type special effects, for which a snow simulant is required to remain airborne for a considerable time so that it can blow the required distance. In addition, a buildup of water and surfactants may occur in the area in which the machine is used, creating a problem with slippery floors and surfaces, as well as staining of floors and surfaces from the foam particles. Furthermore, the generation of aqueous-foam simulated snowflakes requires a powerful airstream to break the foam into small snowflake-size particles, which makes these machines excessively noisy for many theatre, film, or television applications. A further disadvantage of liquid foam type simulated snow is that when viewed close up, for example when the simulated snowfall falls directly on a target audience and they view the material which sticks to their clothes, it looks like foam and lacks the degree of realism desirable for snow special effects.

Simulated snow has also been produced by taking a pre-made flake-like material composed of shredded paper, shredded plastic film, starch flakes, cellulose, or like material, and dispensing it by hand or by the use of a machine. The machines used for this application contain a hopper into which bags or bales of flake-like material are emptied, a mixing arm to break up clumps and render the flake-like material non-agglomerated, and a fan system which blows the flake-like material along a tube to where the snow effect is required. These machines operate in much the same way as commercial machines designed to blow thermal insulation into cavity walls of buildings. One major disadvantage of such machines is that the process of dispensing the product does not increase its volume very much, meaning that an enormous volume of bags or bales of product are required. For a typical film shoot using this type of snow special effect, the bags or bales of product required would fill a sizable road truck. Furthermore, simulated snow of this type is expensive to purchase, the machines used to dispense it are excessively noisy, and clean-up afterwards is time consuming, expensive, and results in a large volume of waste.

Simulated snow has also been produced by the use of a flocculent material generated by the condensation of metaldehyde vapor. The vapor is produced by heating solid metaldehyde, most commonly with heat supplied by combustion of another material. For example, the commercial product known as “snow sticks” or “snow candles” consists of a core of metaldehyde powder surrounded by a tube made from paper impregnated with an oxidizer such as potassium nitrate. On ignition, the impregnated paper tube undergoes a nameless pyrotechnic deflagration which heats the metaldehyde contained therein, producing metaldehyde vapor which condenses in the surrounding atmosphere to produce airborne floccules resembling snow. The floccules have a low density structure containing a high proportion of airspaces, meaning that a small volume of metaldehyde produces a much larger volume of simulated snow. The low density of the product allows it to remain airborne for a considerable time and it can be carried a considerable distance by air currents, making it satisfactory for simulating blizzards or snow drifting in wind. Other special effects products using metaldehyde are known by several names, including “snow tablets” and “snowstorm tablets.” These are small tablets which can be heated by placing on the tip of a burning cigarette, or by other methods. On heating, they produce a small quantity of airborne snow-like floccules which is insufficient for professional special effects applications, but has a striking appearance when suspended in the air of a typical size domestic room. Thus “snow tablets” and “snowstorm tablets” have been sold to the public as a novelty item, commonly with other items collectively known as “indoor fireworks.”

However, metaldehyde is known to exhibit toxicity in humans and animals and is therefore not a desirable chemical for use in special effects products. For this reason, the use of alternative non-toxic compositions are highly desirable. Furthermore, heating of metaldehyde to produce simulated snow generates an unpleasant odor which may limit or preclude its use, for example, commercial “snow sticks” or “snow candles” can generally only be used outdoors.

Only a very small proportion of known materials appear to have physicochemical properties such that, under appropriate conditions, their vapors are able to condense to an airborne flocculent material suitable for application in special effects in place of metaldehyde. It is more common for vapors of substances to produce, upon condensation in air, smoke, mist, fume, dust-like particles, small compact crystals, or droplets. The scientific and technical reasons why the vapors of some materials may condense to flocculent material, whereas other vapors do not, have been little studied and are not well understood by those skilled in the art. For this reason, there is no theoretical basis for predicting which substances are likely to be capable of producing airborne flocculent material upon condensation of the vapor. Thus, serendipity and testing many sublimable or evaporable materials by empirical experiment are the only routes by which those skilled in the art may discover usable replacements for metaldehyde.

Airborne flocculent material generated by the condensation of metaldehyde vapor is white in color. When metaldehyde is mixed with a variety of sublimable or evaporable dyes and heated, either white floc is produced, or floc of such pale color that it looks almost white, possibly because commercially available sublimable or evaporable dyes evaporate and recondense at substantially higher temperatures than metaldehyde. However, there are no known sublimable or evaporable dyes which give satisfactory coloration to metaldehyde-based flocculent material. As such, colored metaldehyde-based flocculent material is not currently commercially available. As such, special effects applications of thermally generated flocculent materials are limited to white snow-type effects.

Flocculent materials in a range of colors can be used to simulate effects including, but not limited to, dust, dirt, ash, and fantasy colored snow. The ability to produce airborne flocculent material in a range of colors from the condensation of vapor is highly desirable in special effects products.

For the reasons listed above, it is desirable to have non-toxic compositions for use in special effects. The present invention describes a non-toxic composition containing amino acids that can be used to produce flocculent material for use in special effects, which can resemble known materials, including but not limited to, flakes of snow, tufts of cotton wool, volcanic ash, or which can have a visual appearance unlike previously known materials. The composition of the invention produces airborne flocculent material which is white in color. The present invention also discloses the optional use of one or more sublimable or evaporable dyes in conjunction with the amino acid composition, to produce flocculent materials having colors, hues, or shades other than white. Additionally, the present invention discloses methods and systems for making flocculent material.

### SUMMARY

The present invention relates to the field of special effects, more particularly production of white or colored flocculent materials, which may resemble known materials including, but not limited to, flakes of snow, tufts of cotton wool, volcanic ash, or materials having a visual appearance unlike previously known materials.

The present invention describes a composition for producing flocculent material, the composition comprising at least one amino acid. The at least one amino acid may be selected from the group comprising alanine, valine, leucine, isoleucine, phenylalanine, proline, cysteine, methionine, serine, threonine, alpha-aminobutyric acid, beta-aminobutyric acid, alpha-aminoisobutyric acid, norvaline, norleucine, homonorleucine, isovaline, or tert-leucine. The amino acids in the composition can be an L-enantiomer, a D-enantiomer, or mixture of L- and D-enantiomers such as, for example, a racemic mixture of enantiomers. The composition can also optionally contain one or more dyes such as, for example, a sublimable or evaporable dye, The one or more optional dyes can be, for example, C.I. Solvent Yellow 33, C.I. Solvent Violet 13, C.I. Solvent Orange 60, C.I. Solvent Red 111, and C.I. Solvent Blue 36, or the like.

The present invention also describes a method for producing flocculent material, the method comprising the steps of: providing a composition with one or more amino acids, heating the composition until a vapor is produced; and cooling the heated vapor-air mixture to produce a flocculent material. In one aspect, the composition is heated between about 225° C. to 350° C.

Also described herein is a system for producing flocculent material, the system comprising a composition with one or more amino acids and a heat source. In one aspect, the heat source comprises a heating plate, which can optionally contain a stirring mechanism. Optionally, an open-topped vessel such as a crucible may be heated by the heat source. Optionally, one or more walls can surround or partially surround the heat source. In another aspect, the system can include a fan. In yet another aspect, the system can include both a fan and a tube.

### DESCRIPTION

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

The terms “about” or “approximately” mean within an acceptable error range for the particular value as determined by one of ordinary skill in the art, which depends in part on how the value is measured or determined. Where particular values are described herein, unless otherwise stated, the term “about” means within an acceptable error range for the particular value. For example, “about” can mean within 1 or more than 1 standard deviation. Alternatively, “about” can mean a range of up to 20%, up to 15%, up to 10%, up to 5%, or up to 1% of a given value. The term can mean within an order of magnitude, preferably within 5-fold and more preferably within 2-fold, of a value.

The terms “determining,” “measuring,” “evaluating,” “assessing,” “assaying,” and “analyzing” are used interchangeably herein to refer to any form of measurement, and include determining if an element is present or not (for example, detection). These terms can include both quantitative and/or qualitative determinations. Assessing may be relative or absolute.

As used herein, the term “comprise” and variations of the term, such as “comprising” and “comprises,” are not intended to exclude other additives, components, integers or steps.

As used herein, the term “amino acid” includes natural or synthetic chemical compounds distinguished by the presence of at least one primary, secondary, or tertiary amine group and at least one carboxylic acid group in each molecule, as well as amino acid analogs and mimetics. An amino acid may have amine and carboxylic acid groups bonded to the same carbon atom, or the amine and carboxylic acid groups may be separated by any number of carbon atoms. An amino acid can be any stereoisomer or mixture of stereoisomers, including D or L enantiomers. An amino acid can be a standard, non-standard, canonical, non-canonical, essential, non-essential, natural, or non-natural amino acid. An amino acid can also have a positively charged side chain, a negatively charged side chain, a polar uncharged side chain, a non-polar side chain, a hydrophobic side chain, a hydrophilic side chain, an aliphatic side chain, an aromatic side chain, a cyclic side chain, an acyclic side chain, a basic side chain, an acidic side chain, or a nucleophilic or electrophilic side chain. An amino acid can have more than one side chain.

The terms “flocculent” or “floc” refers to a form of loosely aggregated particles, soft flakes, or low-density porous material that resembles snow, down, cotton wool, ash, fluff, dust, aerogel, or the like.

The terms “sublimable dye” and “evaporable dye” refers to a dye which, upon heating, produces vapor which, on subsequent cooling, is able to condense to re-form solid dye, or which is able to impart color to substrates which are exposed to the vapor. Exemplary dyes suitable for use in the composition of the invention include, for example, C.I. Solvent Violet 13, C.I. Solvent Orange 60, C.I. Solvent Yellow 33, C.I. Solvent Red 111, C.I. Solvent Blue 36.

Current practice by those skilled in the art of special effects includes simulating airborne drifting snow by using a white flocculent material generated by the condensation of metaldehyde vapor. However, metaldehyde is a toxic material. Additionally, the production of colored metaldehyde-based flocculent material is not currently known in special effects practice.

The present invention relates to compositions, methods, and systems for the production of flocculent material. The present invention discloses a composition containing one or more amino acids, with the optional addition of one or more sublimable or evaporable dyes, to produce white or colored airborne flocculent materials suitable for application in special effects, which are less toxic than metaldehyde. The composition contains at least one standard or non-standard amino acid such as, for example, valine, isoleucine, alanine, leucine, phenylalanine, proline, cysteine, methionine, serine, threonine, alpha-aminobutyric acid, beta-aminobutyric acid, alpha-aminoisobutyric acid, norvaline, norleucine, homonorleucine, isovaline, or tert-leucine. The at least one amino acid used may be any stereoisomer or mixture of stereoisomers, including only an L-enantiomer, only a D-enantiomer, a racemic mixture of equal amounts of L- and D-enantiomer (denoted DL), or any desired mixture of L- and D-enantiomers. The composition optionally contains one or more sublimable or evaporable dyes.

To form the flocculent material, the composition is heated to a temperature at which it sublimes or evaporates and generates a hot vapor. Typically, the temperature used for sublimation or evaporation of the composition is from between about 225° C. to about 350° C.; however, this does not preclude using a temperature outside this range in an embodiment of the invention. Preferably, the temperature used is about 300° C. The temperature used to sublime or evaporate the mixture must be high enough to give a satisfactory rate of sublimation or evaporation and floc production, but not so high as to cause excessive decomposition of the amino acid or dye with consequent undesired effects such as smoke or unpleasant odor. In typical embodiments of the invention, the necessary heat is provided by a heat source such as a hot plate with a electric heating element, fuel burner, or heat-producing chemical composition. Typically, the heating plate is square or rectangular, measuring 2 cm by 4 cm to 30 cm by 30 cm; however, other embodiments of the invention may employ hot plates of shape or size outside this range. The heating plate may have an upper working surface of any desired form, including flat or concave, and any desired surface finish, including smooth or rough textured. Other embodiments of the invention may use a hot plate to heat a vessel such as a crucible, or may use a directly heated vessel in lieu of a heated plate.

In one aspect of the invention, the heat source also contains a stirring mechanism for use in stirring the composition with a stir bar during heating. Typical stirring speeds are between about 200 to about 600 revolutions per minute.

To form the flocculent material, the composition of the invention is heated, either in a container such as, for example, crucible, or heated directly on the heat source. The heated composition sublimes or evaporates into vapor which condenses as it rises and cools in the surrounding air, forming a lightweight, fluffy, airborne flocculent material. This flocculent material may be white, or may have a desired color and/or appearance imparted by the optional presence of the dye. The initially-produced flocculent particles produced can he of varying sizes depending upon the amino acid used in the composition; certain compositions produce very small particles whereas other compositions produce extremely large pieces of flocculent material with dimensions on the order of several centimeters. The airborne flocculent material can also settle onto a surface so that it resembles fallen snow.

In one aspect of the invention, the components to produce the flocculent material of the invention are assembled into a system. The system contains the composition of the invention and a heat source, such as, for example, a hot plate. In one aspect, the heat source also contains a stirring mechanism for use in stirring the composition during heating. The heat source can be contained or partially contained within one or more walls, the one or more walls having one or more openings in the wall. A suitable size one or more openings placed above the heat source allow flocculent material to rise out of the system in the current of warm air. The flocculent material is not appreciably broken up by the gently rising air current, so by the use of an appropriate composition, large elongated pieces of flocculent material may be formed. Depending on the composition used, the flocculent material produced is low in density, falls slowly, and remains airborne for an appreciable time.

The system can also contain a fan such as, for example, an axial or radial fan. The fan is preferably variable in speed. The fan has two functions: it breaks up large pieces of the flocculent material into small pieces which resemble snowflakes; and provides the air movement required to blow the flocculent material where the user desires. The speed of the fan influences the size of the flocculent material. Higher fan speeds produce smaller particles of flocculent material. The optimal fan speed depends on the desired effect of the flocculent material. If a length of tube is attached to the outlet of the system then this will also affect the optimum fan speed.

If the system contains a fan, then one or more tubes can be attached to the system to discharge the flocculent material where the user desires. Tubes may be rigid, flexible, or a tubing system comprising both rigid and flexible components may be employed. When a suitable long tube is used with the system, depending on the composition used, the flocculent material produced is more rounded, looks more like real snowflakes, and is a bit denser and falls more quickly. Use of a long tube is also advantageous in that it allows for the machine to be conveniently positioned, and allows the point of egress of the flocculent material from the tube to be very quiet as it is remote from the other components of the machine, particularly the noise-producing fan. The relative silence of the process is an important feature of the system of the invention.

### EXAMPLES

### Example 1: Composition of the Invention

A composition according the present invention was prepared using DL-valine powder.

### Example 2: Method of Producing Flocculent Material

A thermostatically controlled heating plate was used to produce flocculent material using the composition described above. The temperature was set at 300° C. The composition described in Example 1 was added to the hot heating plate

After 1-10 seconds, the composition began to sublime, and the vapor condensed into large white pieces of flocculent material which rose into the air and remained suspended for some time.

### Example 3: Production of Drifting or Blizzard-Like Snow Effect

To create the effect of blowing snow, an electric axial fan, blowing air upwards, was placed a suitable distance (typically 10-30 cm) above a thermostatically controlled heating plate heated to 300° C. The DL-valine composition described in Example 1 was placed on the plate, which sublimed into a vapor which condensed to large pieces of airborne flocculent material. The large pieces of flocculent material rose into and through the fan, which broke the flocculent material into a size suitable to create drifting or blizzard-like snow-like special effects.

### Example 4: Production of Drifting, Blizzard-Like, or Falling Snow Effect

To create the effect of blowing snow, an electric axial fan, blowing air upwards, was placed a suitable distance (typically 10-30 cm) above a thermostatically controlled heating plate heated to 300° C. A flexible tube 15 cm in diameter and 6 m long tube having a first end and a second end was placed on the outlet of the fan. The DL-valine composition described in Example 1 was placed on the plate, which sublimed into a vapor which condensed to large pieces of airborne flocculent material. The fan broke the large pieces of flocculent material into a smaller size. The smaller flocculent material then went into the first end of the tube, and out of the second end of the tube. The second end of the tube was positioned such that the flocculent material was discharged where desired. By discharging the flocculent material high off the ground a gently falling snow effect was produced. Alternatively, by discharging the flocculent material in front of a large stage fan (also known as a “wind machine”), a blizzard-like effect was produced.

### Example 5: Alternative Composition and Method of Producing Flocculent Material

A composition according to the present invention was prepared using L-valine powder. The composition was placed on a thermostatically controlled heating plate set at 300° C. After 1-10 seconds, the composition began to sublime, and the vapor condensed into small white pieces of flocculent material. The pieces of flocculent material produced were much smaller than those produced by DL-valine and resembled very small snowflakes.

### Example 6: Alternative Composition of the Invention

A composition according the present invention was prepared by grinding together DL-valine and the yellow dye C.I. Solvent Yellow 33, in the approximate proportions of 0.05 g dye for each 1 g of amino acid.

### Example 7: Method of Producing Flocculent Material

A thermostatically controlled heating plate equipped with stirring mechanism was used to produce flocculent material using the composition described in Example 6. The temperature was set at 300° C., and a stirring speed was selected from between 200-600 revolutions per minute. A 55 mL zirconium crucible (approximately 47 mm in diameter and 55 mL high) containing a close-fitting magnetic stirrer bar was placed on the hot heating plate. The composition described in Example 6 was added to the hot crucible, in an amount sufficient to partially cover the base of the crucible.

After a few seconds, the composition began to sublime, and the vapor condensed into pale yellow, airborne flocculent material which was emitted from the mouth of the crucible.

### Example 8: Production of Blizzard-Like Effects

L-Isoleucine was dropped, in amounts typically 50-200 mg at a time, onto a heating plate heated to 300° C. Vapor was produced by the heated L-isoleucine, which condensed in the surrounding atmosphere to small pieces of white flocculent material, suitable to resemble a blizzard-like special effect. The pieces of flocculent material produced were much smaller than those produced by DL-valine and resembled very small snowflakes.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference in their entirety.

What is claimed is:

1. A composition for producing flocculent material, the composition comprising at least one amino acid.

2. The composition of claim 1, wherein the at least one amino acid is selected from the group consisting of alanine, valine, leucine, isoleucine, phenylalanine, proline, cysteine, methionine, serine, threonine, alpha-aminobutyric acid, beta-aminobutyric acid, alpha-aminoisobutyric acid, norvaline, norleucine, homonorleucine, isovaline, or tert-leucine or mixtures thereof.

3. The composition of claim 1, wherein the at least one amino acid is an L-enantiomer, D-enantiomer, or mixture of L- and D-enantiomers.

4. The composition of claim 1, wherein the at least one amino acid comprises valine.

5. The composition of claim 4, wherein the valine comprises a racemic mixture of enantiomers.

6. The composition of claim 1 further comprising one or more dyes.

7. The composition of claim 6, wherein the one or more dyes is a sublimable or evaporable dye.

8. The composition of claim 6, wherein the one or more dyes is selected from the group consisting of C.I. Solvent Yellow 33, C.I. Solvent Violet 13, C.I. Solvent Orange 60, C.I. Solvent Red 111, C.I. Solvent Blue 36, or mixtures thereof.

9. A method for producing flocculent material, the method comprising the steps of:
a. providing the composition of claim 1;
b. heating the composition until a vapor is produced; and
c. cooling the heated vapor to produce a flocculent material.

10. The method of claim 9, wherein the composition is heated between about 225° C. to 350° C.

11. The method of claim 9, wherein the composition consists of at least one amino acid selected from the group comprising alanine, valine, leucine, isoleucine, phenylalanine, proline, cysteine, methionine, serine, threonine, alpha-aminobutric acid, beta-aminobutyric acid, alpha-aminoisobutyric acid, norvaline, norleucine, homonorleucine, isovaline, tert-leucine or mixtures thereof.

12. The method of claim 9, wherein the composition comprises valine.

13. The method of claim 9, wherein the composition comprises isoleucine.

14. The method of claim 9, wherein the composition further comprises one or more dyes.

15. A system for producing flocculent material, the system comprising
a. the composition of claim 1; and
b. a heat source.

16. The system of claim 15, wherein the heat source comprises a heating plate.

17. The system of claim 15, further comprising one or more walls surrounding the heat source.

18. The system of claim 15, further comprising a stir plate.

19. The system of claim 15, further comprising a fan.

20. The system of claim 19, further comprising a tube.
